(19) United States
(12) Patent Application Publication
(10) Pub. No.: US 2022/0073448 A1
(43) Pub. Date: Mar. 10, 2022

(54) METHOD FOR SYNTHESIZING CARBOXY-CONTAINING ANTHRAQUINONE DERIVATIVE, CARBOXY-CONTAINING ANTHRAQUINONE DERIVATIVE PREPARED THEREBY, AND BATTERY SYSTEM COMPRISING SAME

(71) Applicant: CHINA SALT JINTAN CO., LTD., Jiangsu (CN)

(72) Inventors: Yi WU, Jiangsu (CN); Junhui XU, Jiangsu (CN); Juntian HAN, Jiangsu (CN); Zhijun SU, Jiangsu (CN); Liuping CHEN, Jiangsu (CN); Yaoxing CUI, Jiangsu (CN); Dan LI, Jiangsu (CN)

(73) Assignee: CHINA SALT JINTAN CO., LTD., Jiangsu (CN)

(21) Appl. No.: 17/417,771

(22) PCT Filed: Aug. 20, 2020
(86) PCT No.: PCT/CN2020/110212

§ 371 Date: Jun. 24, 2021

(30) Foreign Application Priority Data
Aug. 22, 2019 (CN) 201910777077.2

(51) Int. Cl. C07C 227/16

(52) U.S. Cl. CPC C07C 227/16; C07C 2603/24

(57) ABSTRACT

The present invention provides a method for synthesizing a carboxy-containing anthraquinone derivative, including the following steps: S1, mixing a terminal carboxy-containing dibasic acid with thionyl chloride, and adding toluene as a reaction solvent, followed by adding a catalyst and heating to a predetermined temperature for a reaction; S2, after the reaction is completed, removing the reaction solvent and the thionyl chloride, followed by adding toluene for distillation, to obtain a reactant; S3, mixing the reactant with aminoanthraquinone, adding toluene as a reaction solvent, followed by heating to reflux for a reaction; and S4, after the reaction is completed, removing the reaction solvent, adding a potassium carbonate solution to the residue, filtering it to remove a solid, adjusting the filtrate to a predetermined pH value to precipitate a solid, followed by filtering out, washing, and drying the precipitated solid, to obtain the carboxy-containing anthraquinone derivative.

### BACKGROUND

### Technical Field

The present invention relates to the field of redox flow batteries, and particularly to a method for synthesizing a carboxy-containing anthraquinone derivative, a carboxy-containing anthraquinone derivative prepared thereby and a battery system including the same.

### Description of Related Art

With the rapid development of the human economy, problems such as environmental pollution and energy shortage have become increasingly exacerbated, which has prompted countries around the world to extensively develop and utilize renewable energy sources such as wind, solar, and tidal energy. However, these renewable energy sources are discontinuous, unstable, limited by the geographical environment and difficult to connect to the grid, leading to their low utilization rate, and a high rate of wind and solar power abandoned, thereby wasting resources. Therefore, it is necessary to vigorously develop an efficient, low-cost, safe and reliable energy storage technology that can be used in combination with these renewable energy sources.

Among various electrochemical energy storage strategies, compared to static batteries such as lithium-ion batteries and lead-acid batteries, redox flow batteries (RFBs) have several special technical advantages, such as relatively independent energy and power control, high-current and high-power operation (fast response) and high safety (mainly referring to being non-flammable and non-explosive), and thus are most suitable for large-scale (MW/MWh) electrochemical energy storage. A redox active material is the carrier of energy conversion in a redox flow battery, and it is also the core part in the redox flow battery. Inorganic materials are used as active materials in conventional redox flow batteries (such as vanadium redox flow batteries). However, disadvantages such as high cost, toxicity, limited resources, formation of dendrites, and low electrochemical activity of the inorganic materials limit the large-scale application of redox flow batteries. Organic active materials have drawn widespread attention all over the world due to their advantages, such as low cost, “green,” abundant resources, ease of adjustment of molecular energy levels, and fast electrochemical reactions.

The electrolyte solution used in an aqueous organic redox flow battery has an advantage of being non-flammable, and thus the aqueous organic redox flow battery is safer to operate. In addition, in the aqueous organic redox flow battery, the electrolyte solution has a high conductivity, the electrochemical reaction rate is fast, and the output power is high. Therefore, the aqueous organic redox flow battery becomes a desirable large-scale energy storage technology. At present, the aqueous organic redox flow battery still faces some challenges, such as limited solubility of active materials (organics), electrolyte solutions being liable to cross-contamination, low operating current density, and vulnerability to occur side reactions of water electrolysis. Therefore, the development of a new organic active material to overcome the above disadvantages is of great significance for expanding the chemical space of organic redox flow battery (such as open circuit voltage, energy density and stability).

Anthraquinone is a ubiquitous natural product, which can be extracted from specific plants or artificially synthesized, so it can be produced on a large scale. Replacing inorganic ions in conventional redox flow batteries with anthraquinone-based organics can not only greatly reduce the cost of the battery, but also increase the environmental friendliness of the battery. Moreover, quinone-based materials are structurally designable and have a great potential in the development of redox flow batteries.

### SUMMARY

In view of this, the present invention provides a method for synthesizing a carboxy-containing anthraquinone derivative, which is simple and easy to operate, and low in cost, and can be used in a battery system to solve the problems of electrochemical energy storage.

The present invention further provides a carboxy-containing anthraquinone derivative prepared by the above method.

The present invention further provides an aminoanthraquinone derivative-based redox flow battery system.

The method for synthesizing the carboxy-containing anthraquinone derivative according to an embodiment of a first aspect of the present invention includes the following steps: S1, mixing a terminal carboxy-containing dibasic acid with thionyl chloride, and adding toluene as a reaction solvent, followed by adding a catalyst and heating to a predetermined temperature for a reaction; S2, after the reaction is completed, removing the reaction solvent and the thionyl chloride, followed by adding toluene for distillation, to obtain a reactant; S3, mixing the reactant with aminoanthraquinone, adding toluene as a reaction solvent, followed by heating to reflux for a reaction; and S4, after the reaction is completed, removing the reaction solvent, adding a potassium carbonate solution to the residue, filtering it to remove a solid, adjusting the filtrate to a predetermined pH value to precipitate a solid, followed by filtering out, washing, and drying the precipitated solid, to obtain the carboxy-containing anthraquinone derivative.

In the aminoanthraquinone derivative-based redox flow battery system according to an embodiment of the present invention, a device formed by combining two electrolyte solution reservoirs with a redox flow battery stack is used, and in the redox flow battery stack, a device formed by combining two electrodes, an electrolyzer body, a battery separator, circulation pipelines and circulating pumps is used, and thus the battery system can be applied to the battery environment of a salt cavern system (using electrolyte solutions generated in situ). The battery system has characteristics such as a low cost, readily prepared active material, high safety, and high energy density, stable charging/discharging performance and high solubility of the active material. Meanwhile, it can solve the problems of large-scale (MW/MWh) electrochemical energy storage, and make full use of some abandoned salt cavern (mine) resources.

The method for synthesizing the carboxy-containing anthraquinone derivative according to an embodiment of the present invention further has the following additional technical features.

According to an embodiment of the present invention, in the step S1, the terminal carboxy-containing dibasic acid is one selected from a group consisting of propanedioic acid, butanedioic acid, pentanedioic acid, hexanedioic acid, heptanedioic acid, and octanedioic acid.

According to an embodiment of the present invention, in the step S1, a molar ratio of the terminal carboxy-containing dibasic acid to the thionyl chloride is 1:10, and the reaction is performed for a reaction time of 12 to 24 h.

According to an embodiment of the present invention, in the step S1, the catalyst is one selected from a group consisting of N,N-dimethylformamide, pyridine, N,N-dimethylaniline and caprolactam.

According to an embodiment of the present invention, in the Step S3, the aminoanthraquinone is one selected from a group consisting of 1-aminoanthraquinone, 2-aminoanthraquinone, 1,2-diaminoanthraquinone, 1,4-diaminoanthraquinone, 1,5-diaminoanthraquinone, 1,8-diaminoanthraquinone, and 2,6-diaminoanthraquinone.

According to an embodiment of the present invention, in the step S3, a molar ratio of the aminoanthraquinone to the dibasic acid acylate obtained in the S2 is 1:5, and the reaction is performed for a reaction time of 15 to 24 h.

According to an embodiment of a second aspect of the present invention, the carboxy-containing anthraquinone derivative is prepared by the method for synthesizing the carboxy-containing anthraquinone derivative as described in the above embodiments.

The aminoanthraquinone derivative-based redox flow battery system according to an embodiment of a third aspect of the present invention includes two electrolyte solution reservoirs, the two electrolyte solution reservoirs being arranged to be spaced apart, and respectively being small storage tanks or salt caverns with physical solution-mined cavities formed after mining of a salt mine, wherein electrolyte solutions are stored in the storage tanks or solution-mined cavities, the electrolyte solutions include a positive electrode active material, a negative electrode active material and a supporting electrolyte, the positive electrode active material is potassium ferrocyanide, and the negative electrode active material is the carboxy-containing anthraquinone derivative as described in the above embodiments, the positive electrode active material and the negative electrode active material each are dissolved or dispersed directly in a system with water as a solvent in a bulk form and are respectively stored in the two salt caverns, and the supporting electrolyte is dissolved in the system; and a redox flow battery stack, the redox flow battery stack being in communication with the two electrolyte solution reservoirs, wherein the redox flow battery stack includes an electrolyzer body, the electrolyzer body being filled with the electrolyte solutions; two electrodes, the two electrodes being arranged to face each other; a battery separator, the battery separator being located in the electrolyzer body and being configured to separate the electrolyzer body into a positive electrode zone in communication with a first electrolyte solution reservoir of the two electrolyte solution reservoirs and a negative electrode zone in communication with a second electrolyte solution reservoir of the two electrolyte solution reservoirs, wherein a first electrode of the two electrodes is provided in the positive electrode zone, and a second electrode of the two electrodes is provided in the negative electrode zone, the positive electrode zone contains a positive electrode electrolyte solution including the positive electrode active material, and the negative electrode zone contains a negative electrode electrolyte solution including the negative electrode active material, and the battery separator is configured to be penetrated by the supporting electrolyte and prevent the positive electrode active material and the negative electrode active material from penetrating; current collectors, the current collectors being configured to collect and conduct a current generated by the active material in the redox flow battery stack; circulation pipelines, the circulation pipelines being configured to deliver the electrolyte solution in the first electrolyte solution reservoir into or out of the positive electrode zone, and the circulation pipelines being configured to deliver the electrolyte solution in the second electrolyte solution reservoir into or out of the negative electrode zone; and circulating pumps, the circulating pumps being respectively provided in the circulation pipelines and being configured to supply the electrolyte solutions in a circulation flow.

According to an embodiment of the present invention, the positive electrode active material is one selected from a group consisting of potassium ferrocyanide, sodium ferrocyanide, and ammonium ferrocyanide.

According to an embodiment of the present invention, the positive electrode active material has a concentration of 0.1 to 3.0 mol·L⁻¹, and the negative electrode active material has a concentration of 0.1 to 4.0 mol·L⁻¹.

According to an embodiment of the present invention, the two electrolyte solution reservoirs each are a pressurized sealed container at a pressure of 0.1 to 0.5 MPa.

According to an embodiment of the present invention, an inert gas is introduced into each of the two electrolyte solution reservoirs for purging and maintaining the pressure.

According to an embodiment of the present invention, the inert gas is nitrogen or argon.

According to an embodiment of the present invention, the battery separator includes an anion exchange membrane, a cation exchange membrane, or a polymer porous membrane with a pore size of 10 to 300 nm.

According to an embodiment of the present invention, the supporting electrolyte is at least one selected from a group consisting of a NaCl salt solution, a KCl salt solution, a Na₂SO₄ salt solution, a K₂SO₄ salt solution, a MgCl₂ salt solution, a MgSO₄ salt solution, a CaCl₂) salt solution, and a NH₄Cl salt solution.

According to an embodiment of the present invention, the supporting electrolyte has a molar concentration of 0.1 to 8.0 mol·L⁻¹.

According to an embodiment of the present invention, the electrolyte solution further includes an additive, wherein the additive is potassium hydroxide, and the additive is dissolved in the system to improve solubility of the negative electrode active material.

According to an embodiment of the present invention, the two electrodes each are an electrode made of a carbon material.

According to an embodiment of the present invention, the electrode made of the carbon material includes a carbon felt, carbon paper, carbon cloth, carbon black, activated carbon fiber, activated carbon particle, graphene, graphite felt, or glassy carbon material.

According to an embodiment of the present invention, the two electrodes each have a thickness of 2 to 8 mm.

According to an embodiment of the present invention, each of the current collectors is one selected from a group consisting of an electrically conductive metal plate, a graphite plate and a carbon-plastic composite plate.

### BRIEF DESCRIPTION OF THE DRAWINGS

FIG. 1 shows a schematic structural diagram of an aminoanthraquinone derivative-based redox flow battery system according to an embodiment of the present invention;

FIG. 2 shows a cyclic voltammogram of a 1-[N-(5-carboxybutylacyl)]aminoanthraquinone solution (at a concentration of 2 mM, in a potassium hydroxide aqueous solution at pH=14) according to Embodiment 3 of the present invention at a scan rate of 20 mV/s;

FIG. 3 shows a cyclic voltammogram of a 1-[N-(6-carboxypentylacyl)]aminoanthraquinone solution (at a concentration of 2 mM, in a potassium hydroxide aqueous solution at pH=14) according to Embodiment 4 of the present invention at a scan rate of 20 mV/s;

FIG. 4 shows a cyclic voltammogram of a 1-[N-(7-carboxyhexylacyl)]aminoanthraquinone solution (at a concentration of 2 mM, in a potassium hydroxide aqueous solution at pH=14) according to Embodiment 5 of the present invention at a scan rate of 20 mV/s;

FIG. 5 shows a cyclic voltammogram of a 1-[N-(8-carboxyheptylacyl)]aminoanthraquinone solution (at a concentration of 2 mM, in a potassium hydroxide aqueous solution at pH=14) according to Embodiment 6 of the present invention at a scan rate of 20 mV/s;

FIG. 6 shows a graph of capacity efficiency, voltage efficiency, and energy efficiency of a single battery during 50 cycles according to Embodiment 7 of the present invention;

FIG. 7 is a graph showing changes in relationship between the capacity and voltage of a single battery at the 2nd, 25th, and 50th cycles according to Embodiment 7 of the present invention;

FIG. 8 shows a ¹H NMR spectrum of 1-[N-(6-carboxypentylacyl)]aminoanthraquinone according to an embodiment of the present invention;

FIG. 9 shows a ¹H NMR spectrum of 1-[N-(8-carboxyheptylacyl)]aminoanthraquinone according to an embodiment of the present invention;

FIG. 10 shows a mass spectrum of 1-[N-(6-carboxypentylacyl)]aminoanthraquinone according to an embodiment of the present invention; and

FIG. 11 shows a mass spectrum of 1-[N-(8-carboxyheptylacyl)]aminoanthraquinone according to an embodiment of the present invention.

### REFERENCE NUMERALS IN THE DRAWINGS

aminoanthraquinone derivative-based redox flow battery system 100;
electrolyte solution reservoir 10;
redox flow battery stack 20; two electrodes 21; positive electrode electrolyte solution 22; negative electrode electrolyte solution 23; battery separator 24; circulation pipeline 25; circulating pump 26.

### DESCRIPTION OF THE EMBODIMENTS

Embodiments of the present invention will be described below in detail. Examples of the embodiments are shown in the accompanying drawings, where the same or similar elements, or elements with the same or similar functions are represented by the same or similar reference numerals throughout. The embodiments described below with reference to the accompanying drawings are exemplary, and are only used to explain the present invention, and should not be construed as limiting the present invention.

In the description of the present invention, it should be understood that the orientation or positional relationship indicated by the terms “center,” “longitudinal,” “transverse,” “length,” “width,” “thickness,” “upper,” “lower,” “front,” “rear,” “left,” “right,” “vertical,” “horizontal,” “top,” “bottom,” “inner,” “outer,” “clockwise,” “counterclockwise,” “axial,” “radial,” “circumferential” or the like is based on the orientation or positional relationship shown in the accompanying drawings, and is only for the convenience of describing the present invention and simplifying the description, rather than indicating or implying that the indicated device or element must have a specific orientation, or be configured and operated in a specific orientation, and therefore should not be understood as limiting the present invention. In addition, the features defined by “first” or “second” may explicitly or implicitly include one or more such features. In the description of the present invention, “a plurality of” means two or more, unless otherwise specified.

In the description of the present invention, it should be noted that the terms “installation,” “in connection with” and “in connection to” should be understood in a broad sense, unless otherwise clearly specified and limited. For example, they may be fixed connection, detachable connection, or integral connection; or mechanical connection or electrical connection; or direct connection, or indirect connection through an intermediate medium, or internal communication between two elements. For those of ordinary skill in the art, the specific meaning of the above terms in the present invention can be understood under specific circumstances.

A method for synthesizing a carboxy-containing anthraquinone derivative according to an embodiment of the present invention is described below in detail.

The method for synthesizing the carboxy-containing anthraquinone derivative according to the embodiment of the present invention includes the following steps:

S1, mixing a terminal carboxy-containing dibasic acid with thionyl chloride, and adding toluene as a reaction solvent, followed by adding a catalyst and heating to a predetermined temperature for a reaction;

S2, after the reaction is completed, removing the reaction solvent and the thionyl chloride, followed by adding toluene for distillation, to obtain a reactant;

S3, mixing the reactant with aminoanthraquinone, adding toluene as a reaction solvent, followed by heating to reflux for a reaction; and

S4, after the reaction is completed, removing the reaction solvent, adding a potassium carbonate solution to the residue, filtering it to remove a solid, adjusting the filtrate to a predetermined pH value to precipitate a solid, followed by filtering out, washing, and drying the precipitated solid, to obtain the carboxy-containing anthraquinone derivative.

Specifically, first, acid chlorination of a terminal carboxy-containing dibasic acid is performed as follows. The terminal carboxy-containing dibasic acid and thionyl chloride are mixed and charged into a reactor, then toluene is added thereto as a reaction solvent, and an appropriate amount of a catalyst is added thereto for catalysis, followed by heating to 60° C. for a reaction. After the reaction is completed, the reaction solvent and thionyl chloride are removed by distillation under reduced pressure, and then toluene is added for distillation (20 mL×2), and a residue is used for further reaction. The reactants used in the process are shown below:

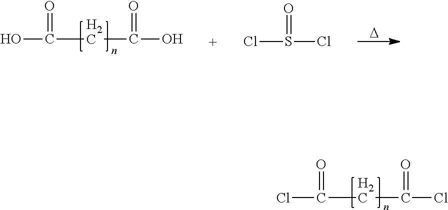

Next, the carboxy-containing aminoanthraquinone is synthesized as follows. The product obtained in the first step and aminoanthraquinone are mixed and charged into a reactor, and then toluene is added thereto as a reaction solvent, followed by heating to reflux for a reaction. After the reaction is completed, the reaction solvent is removed by distillation under reduced pressure, and then a 20% potassium carbonate solution is added to the residue, and being filtered to remove solids. The pH of the filtrate is adjusted (to pH 6) with acetic acid, with a yellow solid being precipitated. The precipitated product is filtered out, washed with hot water (or alcohol), and dried to obtain the target product. The reaction formula is shown below:

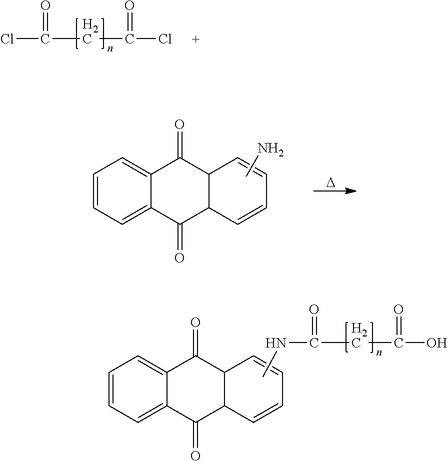

The target product finally obtained has a chemical formula of:

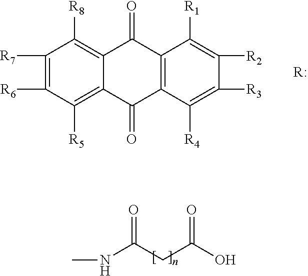

Therefore, the method for synthesizing the carboxy-containing anthraquinone derivative according to the embodiment of the present invention is simple and easy to operate, to readily prepare the active material, and low in cost, and can be used in a battery system to solve the problems of electrochemical energy storage.

According to some particular embodiments of the present invention, in the step S1, the terminal carboxy-containing dibasic acid is one selected from a group consisting of propanedioic acid, butanedioic acid, pentanedioic acid, hexanedioic acid, heptanedioic acid, and octanedioic acid.

Preferably, in the step S1, a molar ratio of the terminal carboxy-containing dibasic acid to the thionyl chloride is 1:10, and the reaction is performed for a reaction time of 12 to 24 h.

Optionally, in the step S1, the catalyst is one selected from a group consisting of N,N-dimethylformamide, pyridine, N,N-dimethylaniline and caprolactam.

According to an embodiment of the present invention, in the step S3, the aminoanthraquinone is one selected from a group consisting of 1-aminoanthraquinone, 2-aminoanthraquinone, 1,2-diaminoanthraquinone, 1,4-diaminoanthraquinone, 1,5-diaminoanthraquinone, 1,8-diaminoanthraquinone, and 2,6-diaminoanthraquinone.

That is, in the chemical formula of the target product, R₁ to R₇ represent the position and number of amino substituents in anthraquinone, and the aminoanthraquinone may be one selected from a group consisting of 1-aminoanthraquinone, 2-aminoanthraquinone, 1,2-diaminoanthraquinone, 1,4-diaminoanthraquinone, 1,5-diaminoanthraquinone, 1,8-diaminoanthraquinone, and 2,6-diaminoanthraquinone. n represents the length of carbon chain in the dicarboxylic acid, and the terminal carboxy-containing dibasic acid may be one selected from a group consisting of propanedioic acid, butanedioic acid, pentanedioic acid, hexanedioic acid, heptanedioic acid, and octanedioic acid.

According to an embodiment of the present invention, in the step S3, a molar ratio of the aminoanthraquinone to the dibasic acid acylate obtained in the S2 is 1:5, and the reaction is performed for a reaction time of 15 to 24 h. Moreover, in the step S1, the catalyst is one selected from a group consisting of N,N-dimethylformamide, pyridine, N,N-dimethylaniline and caprolactam.

A carboxy-containing anthraquinone derivative according to an embodiment of a second aspect of the present invention is prepared by the method for synthesizing the carboxy-containing anthraquinone derivative as described in the above embodiments.

An aminoanthraquinone derivative-based redox flow battery system 100 according to an embodiment of a third aspect of the present invention includes two electrolyte solution reservoirs 10 and a redox flow battery stack 20.

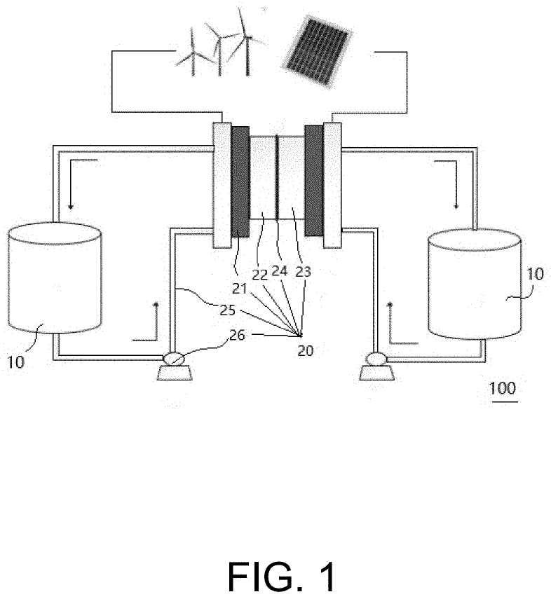

Specifically, as shown in FIG. 1, the two electrolyte solution reservoirs 10 are arranged to be spaced apart. The two electrolyte solution reservoirs 10 are respectively small storage tanks or salt caverns with physical solution-mined cavities formed after mining of a salt mine. Electrolyte solutions are stored in the storage tanks or solution-mined cavities. The electrolyte solutions include a positive electrode active material, a negative electrode active material and a supporting electrolyte. The positive electrode active material is potassium ferrocyanide, and the negative electrode active material is the carboxy-containing anthraquinone derivative according to the above embodiments. The positive electrode active material and the negative electrode active material each are dissolved or dispersed directly in a system with water as a solvent in a bulk form and are respectively stored in the two salt caverns. The supporting electrolyte is dissolved in the system. The redox flow battery stack 20 is in communication with the two electrolyte solution reservoirs 10.

The redox flow battery stack 20 includes an electrolyzer body, two electrodes 21, a battery separator 24, current collectors, circulation pipelines 25 and circulating pumps 26.

Specifically, the electrolyzer body is filled with the electrolyte solutions. The two electrodes 21 are arranged to face each other. The battery separator 24 is located in the electrolyzer body, and the battery separator 24 is configured to separate the electrolyzer body into a positive electrode zone in communication with a first electrolyte solution reservoir 10 of the two electrolyte solution reservoirs 10 and a negative electrode zone in communication with a second electrolyte solution reservoir 10 of the two electrolyte solution reservoirs 10. A first electrode of the two electrodes is provided in the positive electrode zone, and a second electrode of the two electrodes is provided in the negative electrode zone. The positive electrode zone contains a positive electrode electrolyte solution 22 including the positive electrode active material, and the negative electrode zone contains a negative electrode electrolyte solution 23 including the negative electrode active material. The battery separator 24 is configured to be penetrated by the supporting electrolyte and prevent the positive electrode active material and the negative electrode active material from penetrating. The current collectors are configured to collect and conduct a current generated by the active material in the redox flow battery stack 20. The circulation pipelines 25 is configured to deliver the electrolyte solution in the first electrolyte solution reservoir 10 into or out of the positive electrode zone, and the circulation pipelines 25 is configured to deliver the electrolyte solution in the second electrolyte solution reservoir 10 into or out of the negative electrode zone. The circulating pumps 26 are respectively provided in the circulation pipelines 25 and are configured to supply the electrolyte solutions in a circulation flow.

Specifically, the two electrolyte solution reservoirs 10 are arranged to be spaced apart. The two electrolyte solution reservoirs 10 are respectively small storage tanks or salt caverns with physical solution-mined cavities formed after mining of a salt mine. Electrolyte solutions are stored in the solution-mined cavities. The electrolyte solutions include a positive electrode active material, a negative electrode active material and a supporting electrolyte. The positive electrode active material is potassium ferrocyanide, and the negative electrode active material is the novel carboxy-containing aminoanthraquinone derivative. The positive electrode active material and the negative electrode active material each are dissolved or dispersed directly in a system with water as a solvent in a bulk form and are respectively stored in the two salt caverns. The supporting electrolyte is dissolved in the system. The redox flow battery stack 20 is in communication with the two electrolyte solution reservoirs 10. The electrolyzer body is filled with the electrolyte solutions. The two electrodes 21 are arranged to face each other. The battery separator 24 is located in the electrolyzer body, and the battery separator 24 is configured to separate the electrolyzer body into a positive electrode zone in communication with a first electrolyte solution reservoir 10 of the two electrolyte solution reservoirs 10 and a negative electrode zone in communication with a second electrolyte solution reservoir 10 of the two electrolyte solution reservoirs 10. A first electrode 21 of the two electrodes 21 is provided in the positive electrode zone, and a second electrode 21 of the two electrodes 21 is provided in the negative electrode zone. The positive electrode zone contains a positive electrode electrolyte solution 22 including the positive electrode active material, and the negative electrode zone contains a negative electrode electrolyte solution 23 including the negative electrode active material. The battery separator 24 is configured to be penetrated by the supporting electrolyte and prevent the positive electrode active material and the negative electrode active material from penetrating. The circulation pipelines 25 is configured to deliver the electrolyte solution in the first electrolyte solution reservoir 10 into or out of the positive electrode zone, and the circulation pipelines 25 is configured to deliver the electrolyte solution in the second electrolyte solution reservoir 10 into or out of the negative electrode zone. The circulating pumps 26 are respectively provided in the circulation pipelines 25 and are configured to supply the electrolyte solutions in a circulation flow.

In other words, the aminoanthraquinone derivative-based redox flow battery system 100 according to the embodiment of the present invention includes two electrolyte solution reservoirs 10 and a redox flow battery stack 20. The redox flow battery stack 20 includes two electrodes 21, an electrolyzer body, a battery separator 24, circulation pipelines 25 and circulating pumps 26. The two electrolyte solution reservoirs 10 are underground cavities left after solution mining of a salt mine by dissolving salts with water, i.e., salt caverns. Electrolyte solutions are stored in the salt caverns. The electrolyte solutions include a positive electrode active material, a negative electrode active material and a supporting electrolyte. The positive electrode active material is potassium ferrocyanide, and the negative electrode active material is the novel carboxy-containing aminoanthraquinone derivative. The positive electrode active material and the negative electrode active material each are dissolved or dispersed in a system with water as a solvent in a bulk form. The supporting electrolyte is dissolved in the system. The redox flow battery stack 20 is in communication with the two electrolyte solution reservoirs 10 through the circulation pipelines 25. The two electrodes 21 are arranged to face each other. The circulating pumps 26 are respectively provided in the circulation pipelines 25 and are configured to circulate the electrolyte solutions to the two electrodes 21. The two electrodes 21 may be respectively positive electrode and negative electrode. The two electrodes 21 are in direct contact with the electrolyte solutions, respectively, and each provide an electrochemical reaction site with abundant pores. The battery separator 24 is located in the electrolyzer body, and the battery separator 24 is configured to be penetrated by the supporting electrolyte and prevent the positive electrode active material and the negative electrode active material from penetrating. The battery separator 24 may be a cation exchange membrane.

Therefore, in the aminoanthraquinone derivative-based redox flow battery system 100 according to the embodiment of the present invention, a device formed by combining two electrolyte solution reservoirs 10 with a redox flow battery stack 20 is used, and in the redox flow battery stack 20, a device formed by combining two electrodes 21, an electrolyzer body, a battery separator 24, circulation pipelines 25 and circulating pumps 26 is used, and thus the battery system 100 can be applied to the battery environment of a salt cavern system (using electrolyte solutions generated in situ). The battery system 100 has characteristics such as a low cost, readily prepared active material, high safety, and high energy density, stable charging/discharging performance and high solubility of the active material. Meanwhile, it can solve the problems of large-scale (MW/MWh) electrochemical energy storage, and make full use of some abandoned salt cavern (mine) resources.

Preferably, the positive electrode active material is one selected from a group consisting of potassium ferrocyanide, sodium ferrocyanide, and ammonium ferrocyanide.

According to another embodiment of the present invention, the positive electrode active material has a concentration of 0.1 to 3.0 mol·L⁻¹, and the negative electrode active material has a concentration of 0.1 to 4.0 mol·L⁻¹.

Optionally, the two electrolyte solution reservoirs 10 each are a pressurized sealed container at a pressure of 0.1 to 0.5 MPa.

In an embodiment of the present invention, inert gas is introduced into each of the two electrolyte solution reservoirs 10 for purging and maintaining the pressure. The inert gas is introduced into each of the two electrolyte solution reservoirs 10 for protection, and the inert gas can be used for protection all the time during charging and discharging.

Preferably, the inert gas is nitrogen or argon.

In an embodiment of the present invention, the battery separator may be an anion exchange membrane, a cation exchange membrane, or a polymer porous membrane with a pore size of 10 to 300 nm.

According to an embodiment of the present invention, the supporting electrolyte may be at least one selected from a group consisting of a NaCl salt solution, a KCl salt solution, a Na₂SO₄ salt solution, a K₂SO₄ salt solution, a MgCl₂ salt solution, a MgSO₄ salt solution, a CaCl₂) salt solution, and a NH₄Cl salt solution.

According to yet another embodiment of the present invention, the supporting electrolyte has a molar concentration of 0.1 to 8.0 mol·L⁻¹.

Optionally, the electrolyte solution further includes an additive, wherein the additive is potassium hydroxide, and the additive is dissolved in the system to improve the solubility of the negative electrode active material.

According to an embodiment of the present invention, the two electrodes each are an electrode made of a carbon material.

Further, the electrode made of the carbon material includes a carbon felt, carbon paper, carbon cloth, carbon black, activated carbon fiber, activated carbon particle, graphene, graphite felt, or glassy carbon material.

Preferably, the two electrodes each have a thickness of 2 to 8 mm.

Optionally, each of the current collectors is one selected from a group consisting of an electrically conductive metal plate, a graphite plate and a carbon-plastic composite plate.

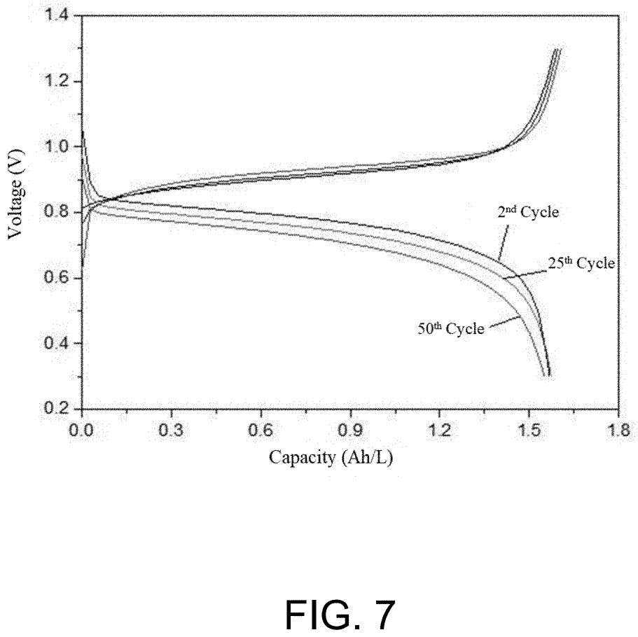

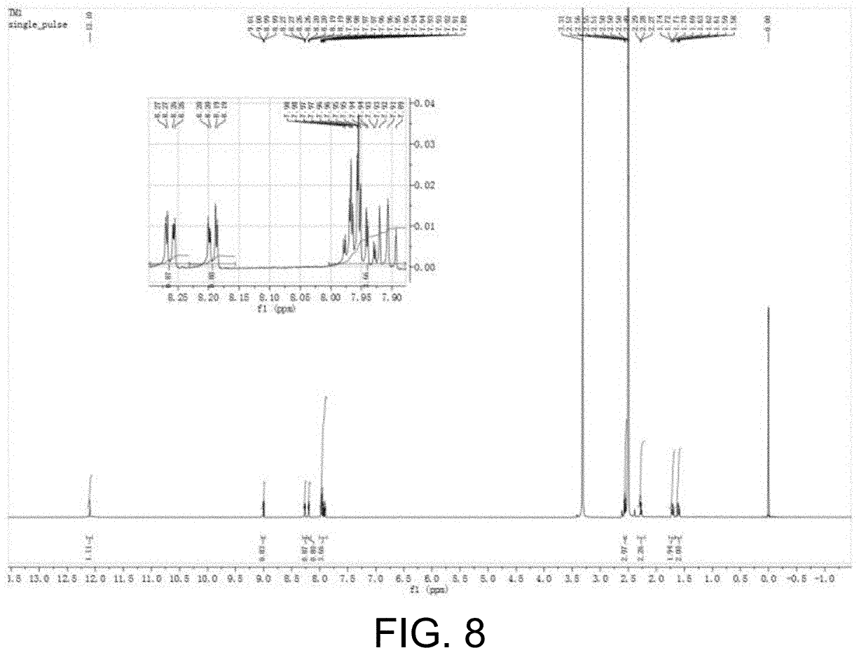

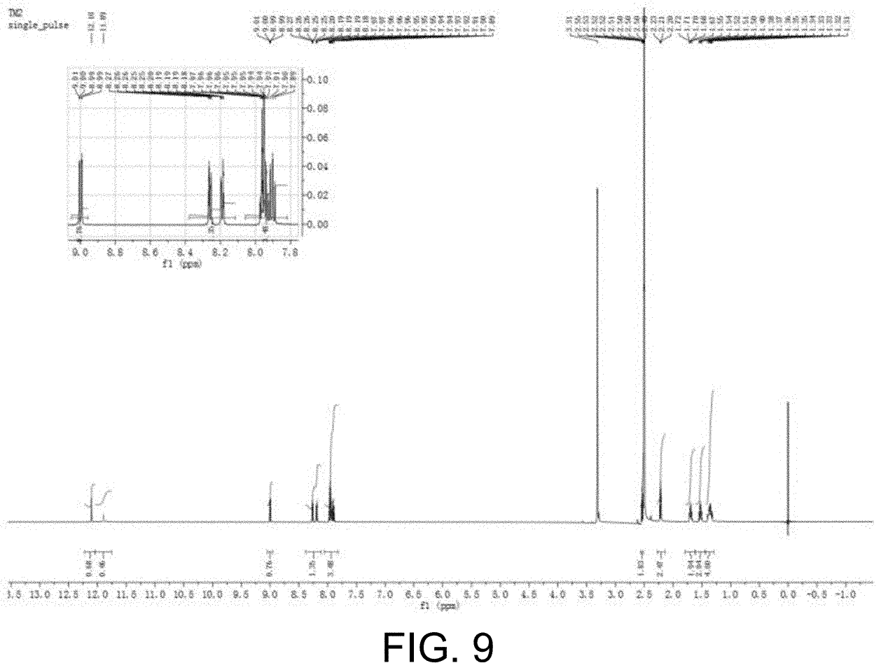

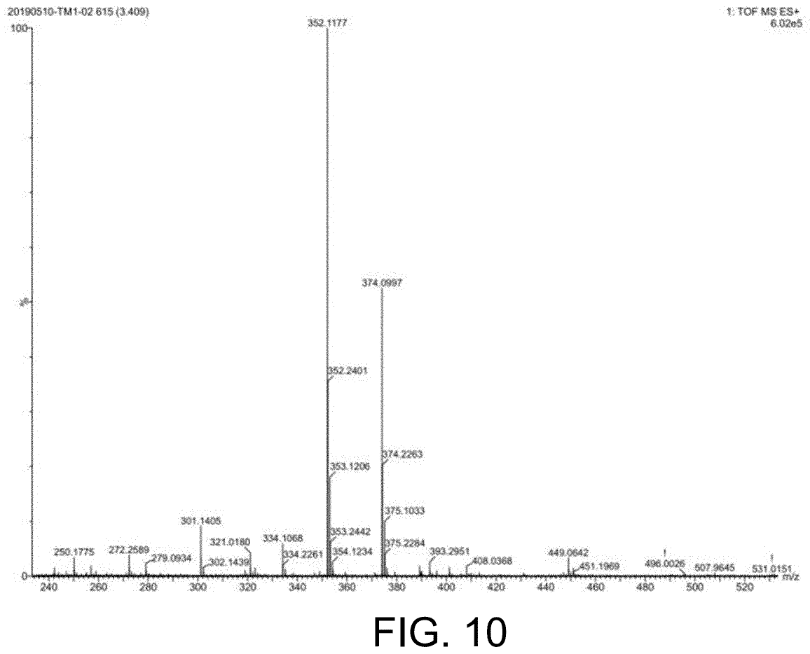

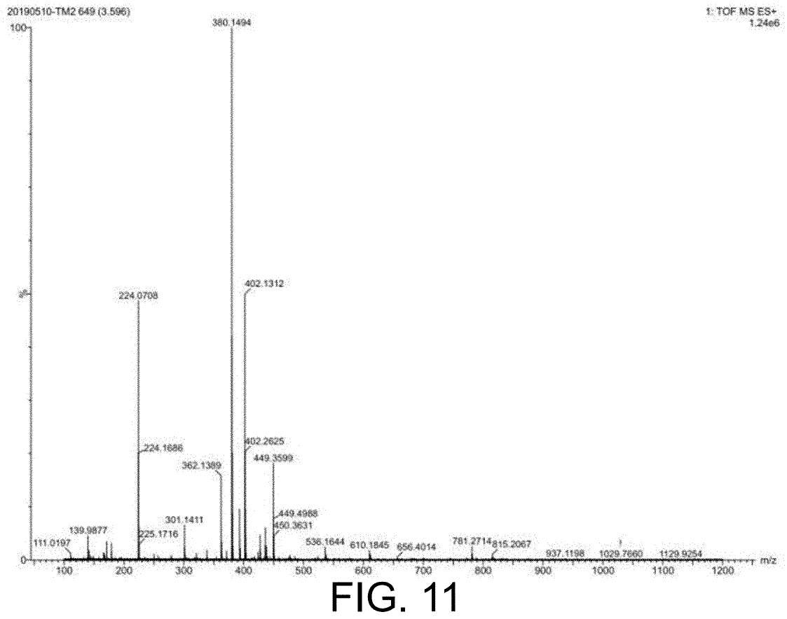

The aminoanthraquinone derivative-based redox flow battery system 100 based on salt caverns according to the embodiments of the present invention will be explained in detail below in combination with particular embodiments and FIGS. 1 to 11.

In the cyclic voltammetry test of the electric pair, the CS Series electrochemical workstation from Wuhan Corrtest Instruments Corp., Ltd. was used to test the electrochemical performance of the organic electric pair with a three-electrode system. The working electrode was a glassy carbon electrode (Tianjin IDA Hengsheng Co.), the reference electrode was a Ag/AgCl electrode, the counter electrode was a platinum electrode, the scan range of the electric pair of positive electrode and negative electrode was −1.0 to 1.0 V, and the scan rate was 20 mV·s⁻¹.

In the battery test, the flow rate of the electrolyte solutions was about 5.0 mL·min⁻¹, and the current density was 80 mA·cm⁻² in the constant current charging/discharging mode.

### Embodiment 1

### Synthesis of 1-[N-(6-carboxypentylacyl)]aminoanthraquinone

2.92 g of hexanedioic acid (0.02 mol) and 15 mL of thionyl chloride were mixed and dissolved in 35 mL of toluene, and 0.01 g of DMF was added thereto as a catalyst, followed by heating to 60° C. for reaction under reflux. When the solvent turned light yellow (12 to 24 h), the reaction was ceased. Thionyl chloride and toluene were removed by distillation under reduced pressure, followed by addition of toluene for distillation (20 mL×2), and a residue was used in the following reaction.

40 mL of toluene and 0.89 g of 1-aminoanthraquinone were added successively to the above residue, followed by slowly raising the temperature to reflux. As the reaction proceeded, the reaction solution gradually turned from red to orange-yellow. The progress of the reaction was monitored by TLC, and the reaction was ceased when the reaction was almost complete (15 to 20 h). The solvent toluene was removed by distillation under reduced pressure (to distill toluene off as completely as possible), and the resulting mixture was dissolved in 200 mL of a sodium carbonate solution (at a concentration of 12%). Unreacted 1-aminoanthraquinone was removed by filtration. Acetic acid was added dropwise to the filtrate, and a light yellow precipitate formed. After complete precipitation, suction filtration was performed and the precipitate was washed with hot water to remove excess 1,6-hexanedioic acid. The product was dried in a vacuum drying oven with a yield of 80%.

### Embodiment 2

### Synthesis of 1-[N-(8-carboxyheptylacyl)]aminoanthraquinone

3.48 g of octanedioic acid (0.02 mol) and 15 mL of thionyl chloride were mixed and dissolved in 35 mL of toluene, and 0.01 g of pyridine was added thereto as a catalyst, followed by heating to 60° C. for reaction under reflux. When the solvent turned light yellow (12 to 24 h), the reaction was ceased. Thionyl chloride and toluene were removed by distillation under reduced pressure, followed by addition of toluene for distillation (20 mL×2), and a residue was used in the following reaction.

40 mL of toluene and 0.89 g of 1-aminoanthraquinone were added successively to the above residue, followed by slowly raising the temperature to reflux. As the reaction proceeded, the reaction solution gradually turned from red to orange-yellow. The progress of the reaction was monitored by TLC, and the reaction was ceased when the reaction was almost complete (15 to 20 h). The solvent toluene was removed by distillation under reduced pressure (to distill toluene off as completely as possible), and the resulting mixture was dissolved in 200 mL of a potassium carbonate solution (at a concentration of 12%). Unreacted 1-aminoanthraquinone was removed by filtration. Acetic acid was added dropwise to the filtrate, and a light yellow precipitate formed. After complete precipitation, suction filtration was performed and the precipitate was washed with alcohol to remove excess 1,8-octanedioic acid. The product was dried in a vacuum drying oven with a yield of 85%.

### Embodiment 3

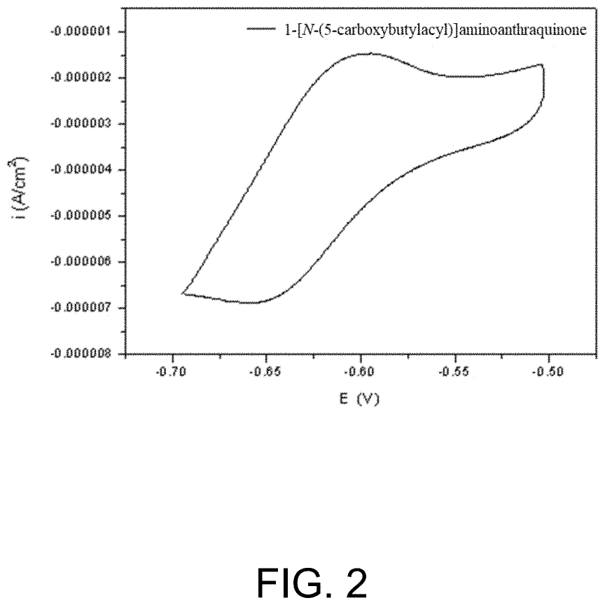

A 1-[N-(5-carboxybutylacyl)]aminoanthraquinone solution (at a concentration of 2 mM, in a potassium hydroxide aqueous solution at pH=14) was investigated by cyclic voltammetry (CV). The CV curve of the compound in FIG. 2 shows redox peaks near −0.65 and −0.60.

### Embodiment 4

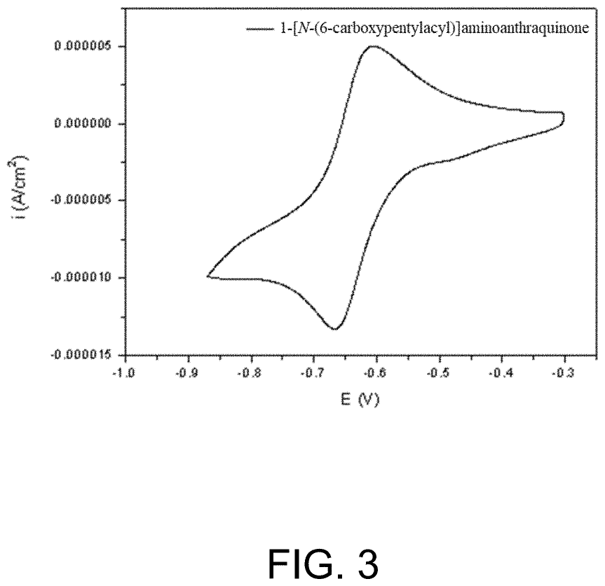

A 1-[N-(6-carboxypentylacyl)]aminoanthraquinone solution (at a concentration of 2 mM, in a potassium hydroxide aqueous solution at pH=14) was investigated by cyclic voltammetry (CV). The CV curve of the compound in FIG. 3 shows redox peaks near −0.66 and −0.60.

### Embodiment 5

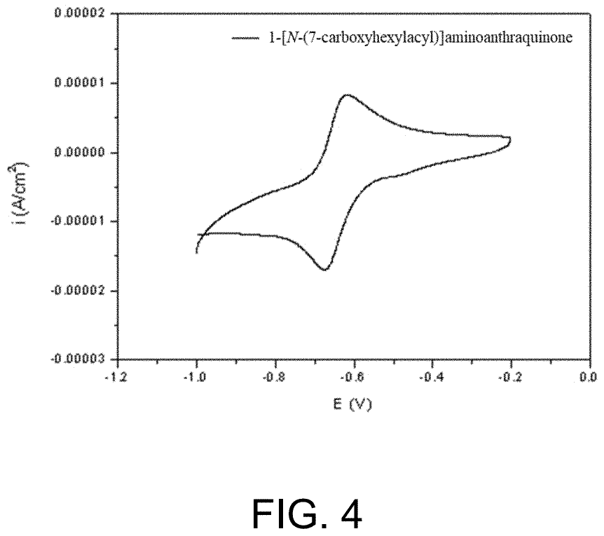

A 1-[N-(7-carboxyhexylacyl)]aminoanthraquinone solution (at a concentration of 2 mM, in a potassium hydroxide aqueous solution at pH=14) was investigated by cyclic voltammetry (CV). The CV curve of the compound in FIG. 4 shows redox peaks near −0.67 and −0.60.

### Embodiment 6

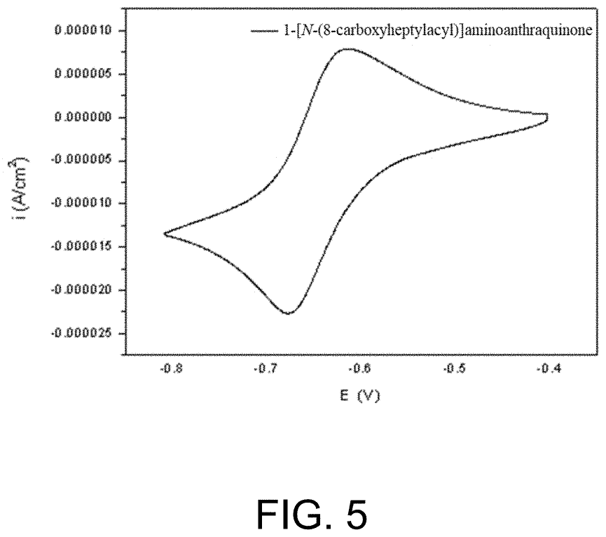

A 1-[N-(8-carboxyheptylacyl)]aminoanthraquinone solution (at a concentration of 2 mM, in a potassium hydroxide aqueous solution at pH=14) was investigated by cyclic voltammetry (CV). The CV curve of the compound in FIG. 5 shows redox peaks near −0.68 and −0.60.

### Embodiment 7

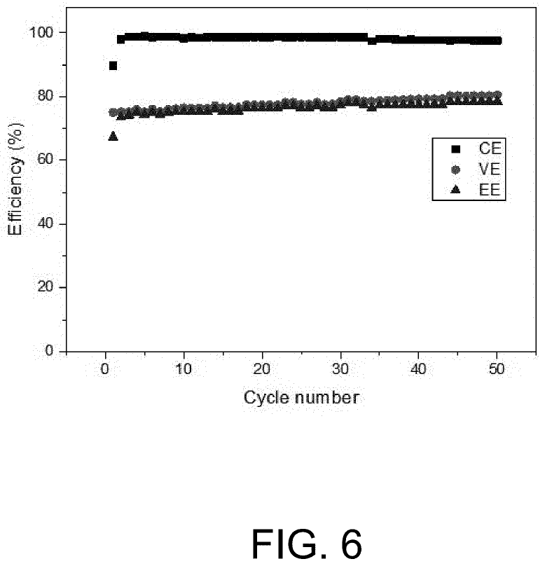

The negative electrode active material in the negative electrode electrolyte solution 23 was 0.1 mol·L⁻¹ 1-[N-(6-carboxypentylacyl)]aminoanthraquinone, and the positive electrode active material in the positive electrode electrolyte solution 22 was 0.2 mol·L⁻¹ K₄Fe(CN)₆. Both the positive electrode electrolyte solution 22 and the negative electrode electrolyte solution 23 comprise a 2.5 mol·L⁻¹ sodium chloride solution as the supporting electrolyte, and the solutions were adjusted to pH 14 with a pH adjusting agent KOH. A single battery of the aqueous system organic redox flow battery system based on salt caverns formed by assembly has a capacity efficiency, voltage efficiency and energy efficiency during 50 cycles of the single battery as shown in FIG. 6. With a cation exchange membrane, at a charge/discharge current of 80 mA/cm², the single battery has a capacity efficiency of 98%, and a voltage efficiency and energy efficiency between 75% and 80%.

In summary, in the aminoanthraquinone derivative-based redox flow battery system 100 according to the embodiments of the present invention, a device formed by combining two electrolyte solution reservoirs 10 with a redox flow battery stack 20 is used, and in the redox flow battery stack 20, a device formed by combining two electrodes 21, an electrolyzer body, a battery separator 24, circulation pipelines 25 and circulating pumps 26 is used, and thus the battery system 100 can be applied to the battery environment of a salt cavern system (using electrolyte solutions generated in situ). The battery system 100 has characteristics such as a low cost, readily prepared active material, high safety, and high energy density, stable charging/discharging performance and high solubility of the active material. Meanwhile, the battery system 100 can solve the problems of large-scale (MW/MWh) electrochemical energy storage, and make full use of some abandoned salt cavern (mine) resources.

The preferred embodiments of the present invention are described above. It should be noted that for those of ordinary skill in the art, several improvements and modifications can be made without departing from the principles of the present invention, and these improvements and modifications should also be regarded as the protection scope of the present invention.

1-7. (canceled)

8. An aminoanthraquinone derivative-based redox flow battery system, comprising:
two electrolyte solution reservoirs, the two electrolyte solution reservoirs being arranged to be spaced apart, and respectively being small storage tanks or salt caverns with physical solution-mined cavities formed after mining of a salt mine, wherein electrolyte solutions are stored in the storage tanks or the solution-mined cavities, the electrolyte solutions comprise a positive electrode active material, a negative electrode active material and a supporting electrolyte, the positive electrode active material is potassium ferrocyanide, and the negative electrode active material is a carboxy-containing anthraquinone derivative, the positive electrode active material and the negative electrode active material each are dissolved or dispersed directly in a system with water as a solvent in a bulk form and are respectively stored in the two salt caverns, and the supporting electrolyte is dissolved in the system; and
a redox flow battery stack, the redox flow battery stack being in communication with the two electrolyte solution reservoirs,
wherein the redox flow battery stack comprises:
an electrolyzer body, the electrolyzer body being filled with the electrolyte solutions;
two electrodes, the two electrodes being arranged to face each other;
a battery separator, the battery separator being located in the electrolyzer body and being configured to separate the electrolyzer body into a positive electrode zone in communication with a first electrolyte solution reservoir of the two electrolyte solution reservoirs and a negative electrode zone in communication with a second electrolyte solution reservoir of the two electrolyte solution reservoirs, wherein a first electrode of the two electrodes is provided in the positive electrode zone, and a second electrode of the two electrodes is provided in the negative electrode zone, the positive electrode zone contains a positive electrode electrolyte solution comprising the positive electrode active material, and the negative electrode zone contains a negative electrode electrolyte solution comprising the negative electrode active material, and the battery separator is configured to be penetrated by the supporting electrolyte and prevent the positive electrode active material and the negative electrode active material from penetrating;
current collectors, the current collectors being configured to collect and conduct a current generated by the positive electrode active material and the negative electrode active material in the redox flow battery stack;
circulation pipelines, a first circulation pipelines of the circulation pipelines being configured to deliver the positive electrode electrolyte solution in the first electrolyte solution reservoir into or out of the positive electrode zone, and a second circulation pipelines of the circulation pipelines being configured to deliver the negative electrode electrolyte solution in the second electrolyte solution reservoir into or out of the negative electrode zone; and
circulating pumps, the circulating pumps being respectively provided in the circulation pipelines and being configured to supply the electrolyte solutions in a circulation flow,
wherein a method for synthesizing the carboxy-containing anthraquinone derivative comprises the following steps:
step S1, mixing a terminal carboxy-containing dibasic acid with thionyl chloride to obtain a first mixture, and adding toluene as a reaction solvent to the first mixture, followed by adding a catalyst and heating to a predetermined temperature for a reaction:
step S2, after the reaction is completed to obtain a first resultant, removing the reaction solvent and the thionyl chloride from the first resultant, followed by adding toluene for distillation, to obtain a reactant;
step S3, mixing the reactant with aminoanthraquinone to obtain a second mixture, and adding toluene as a reaction solvent to the second mixture, followed by heating to reflux for a reaction; and
step S4, after the reaction is completed to obtain a second resultant, removing the reaction solvent from the second resultant to obtain a residue, adding a potassium carbonate solution to the residue to obtain a suspension, filtering the suspension to remove a solid and obtain a filtrate, adjusting the filtrate to a predetermined pH value to precipitate a solid, followed by filtering out, washing, and drying the precipitated solid, to obtain the carboxy-containing anthraquinone derivative.

9. The aminoanthraquinone derivative-based redox flow battery system according to claim 8, wherein the positive electrode active material is one selected from a group consisting of potassium ferrocyanide, sodium ferrocyanide, and ammonium ferrocyanide.

10. The aminoanthraquinone derivative-based redox flow battery system according to claim 8, wherein the positive electrode active material has a concentration of 0.1 to 3.0 mol·L⁻¹, and the negative electrode active material has a concentration of 0.1 to 4.0 mol·L⁻¹.

11. The aminoanthraquinone derivative-based redox flow battery system according to claim 8, wherein the two electrolyte solution reservoirs each are a pressurized sealed container at a pressure of 0.1 to 0.5 MPa.

12. The aminoanthraquinone derivative-based redox flow battery system according to claim 8, wherein an inert gas is introduced into each of the two electrolyte solution reservoirs for purging and maintaining a pressure.

13. The aminoanthraquinone derivative-based redox flow battery system according to claim 12, wherein the inert gas is nitrogen or argon.

14. The aminoanthraquinone derivative-based redox flow battery system according to claim 8, wherein the battery separator comprises an anion exchange membrane, a cation exchange membrane, or a polymer porous membrane with a pore size of 10 to 300 nm.

15. The aminoanthraquinone derivative-based redox flow battery system according to claim 8, wherein the supporting electrolyte is at least one selected from a group consisting of a NaCl salt solution, a KCl salt solution, a Na₂SO₄ salt solution, a K₂SO₄ salt solution, a MgCl₂ salt solution, a MgSO₄ salt solution, a CaCl₂ salt solution, and a NH₄Cl salt solution.

16. The aminoanthraquinone derivative-based redox flow battery system according to claim 8, wherein the supporting electrolyte has a molar concentration of 0.1 to 8.0 mol·L⁻¹.

17. The aminoanthraquinone derivative-based redox flow battery system according to claim 8, wherein the negative electrode electrolyte solution further comprises an additive, wherein the additive is potassium hydroxide, and the additive is dissolved in the system to improve solubility of the negative electrode active material.

18. The aminoanthraquinone derivative-based redox flow battery system according to claim 9, wherein the two electrodes each are an electrode made of a carbon material.

19. The aminoanthraquinone derivative-based redox flow battery system according to claim 18, wherein the electrode made of the carbon material comprises a carbon felt, carbon paper, carbon cloth, carbon black, activated carbon fiber, activated carbon particle, graphene, graphite felt, or glassy carbon material.

20. The aminoanthraquinone derivative-based redox flow battery system according to claim 8, wherein the two electrodes each have a thickness of 2 to 8 mm.

21. The aminoanthraquinone derivative-based redox flow battery system according to claim 8, wherein each of the current collectors is one selected from a group consisting of an electrically conductive metal plate, a graphite plate and a carbon-plastic composite plate.
